SEQUENCE LISTING (1 sequence)

SEQ ID NO: 1 - DNA, 21 bp, synthetic construct
  source 1..21: mol_type = other DNA
agatgtcttgtgctgccggta

(19) United States
(12) Patent Application Publication
(10) Pub. No.: US 2025/0062038 A1
(43) Pub. Date: Feb. 20, 2025

(54) METHOD OF DETECTION OF SEVERE ACUTE RESPIRATORY SYNDROME (SARS)-COVID (SARS-CoV)

(71) Applicant: KING FAHD UNIVERSITY OF PETROLEUM AND MINERALS, Dhahran (SA)

(72) Inventors: Amjad Bajes KHALIL, Dhahran (SA); Muhammad NAEEM, Dhahran (SA); Hanoof Fahad ALKHODAIRY, Riyadh (SA); Aasif HELAL, Dhahran (SA)

(73) Assignee: KING FAHD UNIVERSITY OF PETROLEUM AND MINERALS, Dhahran (SA)

(21) Appl. No.: 18/451,410

(22) Filed: Aug. 17, 2023

(51) Int. Cl. G16H 50/80; G01N 33/58

(52) U.S. Cl. CPC G16H 50/80; G01N 33/582

(57) ABSTRACT

A method of detecting SARS-COV in a sample includes contacting a metal-organic framework with at least one fluorophore-labeled single-stranded probe deoxyribose nucleic acid (DNA) to form a biosensor. The metal-organic framework of the method is zeolitic imidazolate framework-8 (ZIF-8), and the fluorophore-labeled single-stranded probe DNA (p-DNA) has a fluorescence signal at 513 to 517 nm. The method further includes contacting the sample with the biosensor in a solution, and the sample comprises a target sequence of SARS-COV. The target sequence of SARS-COV and the fluorophore-labeled single-stranded p-DNA of the method hybridize to form a double-stranded product. Following the step of the hybridization forming the double-stranded product, the method includes detecting the double-stranded product by observing a change in fluorescence.

### STATEMENT REGARDING PRIOR DISCLOSURE BY THE INVENTORS

Aspects of the present disclosure were described in an article titled “Development of Metal organic Framework Based Biosensor to Detect the Coronavirus (Covid-19)” published in bioRxiv on Aug. 22, 2023, which is incorporated herein by reference in its entirety.

### REFERENCE TO SEQUENCE LISTING

In accordance with 37 CFR § 1.52 (e) (5) and with 37 CFR § 1.831, the specification makes reference to a Sequence Listing submitted electronically as a .xml file named “547183US ST26”. The .xml file was generated on Jul. 3, 2023 and is 4,096 bytes in size. The entire contents of the Sequence Listing are hereby incorporated by reference.

### BACKGROUND

### Technical Field

The present disclosure is directed to a method of detection of severe acute respiratory syndrome coronavirus (SARS-COV), and particularly to a method for detecting the SARS-COV in a sample using a metal organic framework-based biosensor.

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly nor impliedly admitted as prior art against the present invention.

Coronavirus disease 2019, or COVID-19, was first reported in 2019 and soon led to a worldwide pandemic causing millions of deaths and more than a hundred million detected and undetected infections. The COVID-19 pandemic has had a catastrophic effect on human lives all over the world, emerging as a major global health crisis. To contain the rapid increase and spread of COVID-19 infections caused by the SARS-COV virus or COVID-19 virus, timely testing of symptomatic and asymptomatic persons has been supported to be the most effective measure globally. More than three years have gone by since the COVID-19 infection was detected for the first time, and the world has yet to develop a single potent vaccine to curb all variants of the COVID-19 virus. Although many countries have brought the COVID-19 infections and spread under control, recent outbreaks in China and India have raised an alarm reaffirming the World Health Organization's (WHO) Health Committee remark that the COVID-19 disease is here to stay, and the emergence of new variants will continue to cause new surges in cases of the infections and deaths. As per the Director Genreal of WHO, the COVID-19 virus and infection still claims a life every three minutes [United Nations, 5 May 2023]. Thus, COVID-19 is set to remain an active infection among human populations, and an outbreak of the infection will remain a challenge for generations to come. Such a scenario necessitates the need for a robust detection method and a rapid testing system for early and accurate detection to contain the spread of the disease now and in the future. Another challenge posed by the COVID-19 infection is that the disease can be transmitted to others by asymptomatic individuals, and an accurate and reliable testing system will remain the first line of defense against any future COVID-19 outbreaks.

Advancements have been brought about for the detection and diagnosis of the COVID-19 infection since the pandemic began. Broadly, two types of tests have been extensively developed and considered for early detection and diagnosis of the disease. According to the Center for Disease Control and Prevention (CDC), the test types used to detect the COVID-19 infection include viral tests such as PCR tests, antigen tests, and breath tests to detect COVID-19 infections. Results of these tests determine the need for prevention measures, such as isolation and quarantine. Viral tests are useful for screening for infected individuals; however, viral tests, such as PCR tests, require advanced laboratory equipment and, to some extent, can only be performed at point-of-care. Tests such as antigen tests detect a presence of specific viral proteins called antigens. Antigen tests are rapid and are used for determining current infections.

Another category of tests is antibody or serological testing, employed mainly for surveillance and determining epidemiological scenarios. Antibody testing detects specific antibodies in the blood that target specific viruses. The antibody testing methodology is not as common for the detection of infections.

While availability of multiple testing methods is encouraging in a situation such as a pandemic, most of the methods mentioned above and presently utilized come with several drawbacks posing serious healthcare concerns. The more preferred tests, such as PCR tests, require advanced equipment and have a long turnaround time. Certain equipment, such as a thermal cycle, is mandatory for performing PCR. A percentage of false negative results have been seen in the detection of COVID-19 infections using the PCR testing method. Antigen tests, though rapid, have a higher rate of false negative results compared to the PCR tests. An additional concern regarding the serology immunoassays is the lack of the necessary accuracy to be a reliable SARS-CoV diagnostic test. The serology immunoassays also show low sensitivity and are less reliable.

A major challenge the COVID-19 virus has posed before the scientific community is the rate of mutations that the COVID-19 virus has underwent since the pandemic began. Vaccines and drugs may have limited effectiveness against possible mutants of the virus. Diagnosis remains a factor in containing the spread of COVID-19 and preventing further spread of infections. As described above, the current approaches show drawbacks such as low sensitivity and low accuracy. Furthermore, the need for sample preparation and purification methods is labor intensive. Additionally, the use of cumbersome instruments and equipment maintenance comes at a high cost. While the operation of such instruments is complex, a need for qualified personnel is also apparent.

Hence, early identification and isolation of infected individuals is apparent for the success of COVID-19 control. To address such a scenario, a reliable, portable, rapid, user-friendly, and low-cost method is a prerequisite to address the outbreaks of infectious diseases. Thus, there is a need for an accurate, simple, sensitive, cost-effective, portable, scalable, and broadly applicable method and system to handle a pandemic such as COVID-19. Accordingly, an object of the present disclosure is to provide a method of SARS-COV detection that can overcome limitations of the art.

### SUMMARY

In an exemplary embodiment, a method of detecting SARS-COV in a sample is disclosed. The method includes contacting a metal-organic framework with at least one fluorophore-labeled single-stranded probe deoxyribose nucleic acid (DNA) to form a biosensor. The metal-organic framework of the method is zeolitic imidazolate framework-8 (ZIF-8) and the fluorophore-labeled single-stranded probe DNA has a fluorescence signal at 513 to 517 nm in the absence of ZIF-8. The fluorophore-labeled single-stranded probe DNA docks with the metal-organic framework through attractive noncovalent π-orbital overlaps, and the biosensor is fluorescently inactive. The method further includes a step of contacting the sample with the biosensor in a solution. The sample comprises a target sequence of SARS-COV. The target sequence of SARS-COV and the fluorophore-labeled single-stranded probe DNA hybridize to form a double-stranded product. The method includes detecting the double-stranded product by observing a change in fluorescence. The change in fluorescence is a re-emergent fluorescence signal at 513 to 517 nm of the fluorophore-labeled single-stranded probe DNA, and the re-emergent fluorescence signal at 513 to 517 nm of the fluorophore-labeled single-stranded probe DNA indicates the presence of SARS-CoV in the sample, thereby detecting SARS-COV in the sample.

In some embodiments, the fluorophore-labeled single-stranded probe DNA is complementary to the target sequence of SARS-COV.

In some embodiments, a fluorophore of the fluorophore-labeled single-stranded probe DNA is a carboxyfluorescein. In a specific example, the carboxyfluorescein is a 6-carboxyfluorescein moiety. In other examples, the fluorophore of the fluorophore-labeled single-stranded probe DNA is tagged at a 5′ end of the fluorophore-labeled single-stranded probe DNA.

In some embodiments, the target sequence of SARS-COV is an RNA target sequence. In some other examples, the target sequence of SARS-COV is a conserved sequence. In a specific embodiment, the SARS-COV is SARS-COV-2.

In some examples of the present disclosure, the ZIF-8 comprises zinc and 2-methylimidazole and was synthesized at room temperature with an at least 75% yield based on a starting weight of zinc salt and 2-methylimidazole. In an embodiment, the ZIF-8 has a thermogravimetric analysis stability of up to 510° C.

In some examples of the above method, a lower limit of detection of the SARS-COV is a concentration of 200 pM of the target sequence of SARS-COV in the sample.

In some embodiments, the solution comprises a tris-acetate-ethylenediaminetetraacetic acid (TAE) buffer comprising a tris(hydroxymethyl)aminomethane base, an acetic acid, and an ethylenediaminetetraacetic acid.

In some embodiments, the fluorophore-labeled single-stranded probe DNA docks with the metal-organic framework through attractive noncovalent π-orbital overlaps of at least one imidazole the 2-methylimidazole of the metal-organic framework and at least one fluorophore of the fluorophore-labeled single-stranded probe DNA.

In some embodiments of the present method, a ratio of the fluorophore-labeled single-stranded probe DNA to the ZIF-8 in the biosensor is from 1:10 to 1:50 by weight.

In some embodiments, the re-emergent fluorescence signal at 513 to 517 nm of the fluorophore-labeled single-stranded probe DNA is at least 90% of an initial fluorescence signal at 513 to 517 nm of the fluorophore-labeled single-stranded probe DNA in the absence of ZIF-8.

In some embodiments, the contacting the sample with the biosensor in the solution is at a temperature of 0 to 10° C. In some embodiments, the method includes contacting the sample with the biosensor in the solution for a time of 20 to 40 minutes.

In some embodiments, the target sequence of SARS-COV is at least 85% complementary to the fluorophore-labeled single-stranded probe DNA, and the re-emergent fluorescence signal at 513 to 517 nm of the fluorophore-labeled single-stranded probe DNA is from 15 to 95% of an initial fluorescence signal at 513 to 517 nm of the fluorophore-labeled single-stranded probe DNA in the absence of ZIF-8.

In some embodiments, fluorophore-labeled single-stranded probe DNA comprises SEQ ID NO.: 1.

In some aspects of the present disclosure, the method of detecting SARS-COV in a sample is described. The method includes detecting one or more RNA viruses and one or more DNA viruses in the sample.

These and other aspects of the non-limiting embodiments of the present disclosure will become apparent to those skilled in the art upon review of the following description of specific non-limiting embodiments of the disclosure in conjunction with the accompanying drawings. The foregoing general description of the illustrative present disclosure and the following detailed description thereof are merely exemplary aspects of the teachings of this disclosure and are not restrictive.

### BRIEF DESCRIPTION OF DRAWINGS

A complete appreciation of this disclosure (including alternatives and/or variations thereof) and many of the attendant advantages thereof will be readily obtained as the same becomes better understood by reference to the following detailed description when considered in connection with the accompanying drawings, wherein:

FIG. 1 is a flowchart depicting a method of detecting SARS-COV in a sample, according to certain embodiments;

FIG. 2 shows a schematic representation of zeolite imidazolate framework (ZIF)-8/probe-DNA (p-DNA) system as a biosensor to detect coronavirus (COVID-19) with (a) showing fluorescence of p-DNA; (b) showing fluorescence quenching of p-DNA by adding ZIF-8 and (c) showing fluorescence re-emergence by adding TO COVID RNA into the ZIF-8/p-DNA complex., according to certain embodiments;

FIG. 3 shows a schematic representation of ZIF-8 metal-organic framework (MOF) synthesis, according to certain embodiments;

FIG. 4 depicts a powder X-ray diffraction (PXRD) of ZIF-8, and ZIF-8+p-DNA+T0, according to certain embodiments;

FIG. 5 shows thermogravimetric analysis (TGA) analysis of the ZIF-8, according to certain embodiments;

FIG. 6 depicts gradual fluorescence quenching of the p-DNA after adding various concentration of ZIF-8, sequentially, according to certain embodiments;

FIG. 7 depicts fluorescence intensities of the p-DNA (150 nM) alone and in combination with ZIF-8, 2-methyleimidazole, and zinc nitrate in 20 mM tris-acetate-ethylenediaminetetraacetic acid (TAE) buffer at room temperature, according to certain embodiments;

FIG. 8 depicts fluorescence re-emergence of the p-DNA, p-DNA/ZIF-8, p-DNA/zinc nitrate, p-DNA/ZIF-8 with TO, and p-DNA/zinc nitrate with TO, in 20 mM TAE buffer at 4° C. at varying concentrations, according to certain embodiments;

FIG. 9 depicts fluorescence re-emergence of the p-DNA, p-DNA/ZIF-8, p-DNA/ZIF-TO, p-DNA/ZIF-T1, and p-DNA/ZIF-T2, with different varying concentrations, according to certain embodiments;

FIG. 10 depicts a Fourier transform infrared (FTIR) spectroscopy of the ZIF-8, according to certain embodiments;

FIG. 11 depicts a nitrogen (N₂) adsorption isotherm data of the ZIF-8, according to certain embodiments; and

FIG. 12 depicts a field emission scanning electron microscopy (FESEM) image of ZIF-8, according to certain embodiments.

### DETAILED DESCRIPTION

In the following description, it is understood that other embodiments may be utilized, and structural and operational changes may be made without departure from the scope of the present embodiments disclosed herein.

Reference will now be made to specific embodiments or features, examples of which are illustrated in the accompanying drawings. In the drawings, whenever possible, corresponding or similar reference numerals will be used to designate identical or corresponding parts throughout the several views. Moreover, references to various elements described herein are made collectively or individually when there may be more than one element of the same type. However, such references are merely exemplary in nature. It may be noted that any reference to elements in the singular may also be constructed to relate to the plural and vice-versa without limiting the scope of the disclosure to the exact number or type of such elements unless set forth explicitly in the appended claims. Further, as used herein, the words “a,” “an,” and the like generally carry a meaning of “one or more,” unless stated otherwise.

Furthermore, the terms “approximately,” “approximate,” “about,” and similar terms generally refer to ranges that include the identified value within a margin of 20%, 10%, or preferably 5%, and any values therebetween.

As used herein, “sample” refers to any material or mixture of materials containing one or more analytes or entities of interest. As used herein, “analyte” refers to any substance that is suitable for testing in the present disclosure. The sample can be derived from a human patient or an animal, e.g., bodily fluids (blood, nasal secretions, saliva, urine, etc.), biopsy, tissue, and/or waste from the patient. Thus, tissue biopsies, stool, sputum, saliva, blood, lymph, tears, sweat, urine, nasal secretions, or the like can be used in the method, as can essentially any tissue of interest.

As used herein, “metal-organic framework” or “MOF” includes a class of porous materials consisting of linkers coordinated to metals resulting in a one-dimensional (1D), two-dimensional (2D), or three-dimensional (3D) structure with well-defined and repeated structural characteristics throughout the material. A 1D MOF structure may include nanoribbons, nanorods, and/or nanospheres, and the like. A 2D MOF structure may include planar, sheet-like structures. A 3D MOF structure may include spheres, cubes, cylinders, repeating units of 1D MOF structures and 2D MOF structures, and the like.

As used herein, “fluorescence” refers to the emission of light by a substance that has absorbed light or other electromagnetic radiation and may include luminescence, photoluminescence, fluorescence, and phosphorescence.

The term “biosensor”, as used herein, refers to an analytic device comprising a biological component and a physiochemical detector. The biological component may be a tissue, a microorganism, a cell receptor, an enzyme, an antibody, a nucleic acid, a combination thereof, and the like. The biological component is a biologically derived material or biomimetic component that interacts with, binds with, or recognizes an analyte. The analyte may be a chemical compound that is of interest in an analytical procedure. The biological component may comprise a biological receptor (bioreceptor). The bioreceptor may be one or more of an enzyme, an antibody, a cell, a nucleic acid, an aptamer, a portion thereof, a combination thereof, and the like. The physiochemical detector, or transducer, transforms one signal (e.g., chemical or physical information) into another signal (e.g., an analytical signal). The physiochemical detector may work in an optical, piezoelectric, electrochemical, electrochemiluminescent way, the like, and a combination thereof as a result of an interaction of the analyte with the biological component. Various types of biosensors are known in the art. Such as, electrochemical biosensors based on the reaction of enzymatic catalysis that consumes or generates electrons and includes, e.g., amperometric biosensors, potentiometric biosensors, impedimetric biosensors, and voltammetric biosensors.

The term “nucleic acid”, as used herein, refers to either deoxyribonucleic acid (DNA), ribonucleic acid (RNA), single-stranded or double-stranded, and any chemical modifications thereof. The nucleic acid detected according to the systems, assays, and/or methods disclosed herein may be a full-length nucleic acid or a fragment thereof. Nucleic acids are biopolymers and macromolecules comprising nucleotides. Nucleotides are monomer components comprising a five-carbon sugar, a phosphate group, and a nitrogenous base. The nitrogenous base, or nucleobase, may be adenine “A”, cytosine “C”, guanine “G”, thymine “T”, uracil “U”, and any nitrogenous base known in the art.

The terms “sensitivity” and “specificity”, as used herein, have the meaning commonly understood in the art (see e.g., T. Fawcett, “An introduction to ROC analysis”, 2006, 27, 8, 861-874, incorporated herein by reference in its entirety).

A “probe” herein used includes a nucleic acid sequence and/or a nucleic acid sequence analogue, such as, but not limited to, a DNA, an RNA, or a peptide nucleic acid (PNA) sequence, complementary to and capable of hybridizing to a target nucleic acid sequence.

As used herein, “target nucleic acid” or “target” refers to a nucleic acid sequence to be analyzed or detected (e.g., a nucleic acid to which a probe is complementary to and hybridizes). The term refers to both the subsequence to which the probe is directed and to a larger nucleic acid sequence, including the subsequence.

As used herein, the term “hybridize” refers to a process of formation of double stranded DNA regions between one, two, or many single stranded DNA molecules complementary to one, two, or many target nucleic acid sequences. Hybridization may occur through specific hydrogen bonds between standard (Watson-Crick) base pair(s).

Nucleic acid(s) that are “complementary” or “complement(s)” are those that are capable of base-pairing according to the standard Watson-Crick, Hoogsteen, or reverse Hoogsteen binding complementarity rules.

As used herein, the term “complementary” or “complement(s)” may refer to nucleic acid(s) that are substantially complementary, as may be assessed by the same nucleotide comparison set forth above. The term “substantially complementary” may refer to a nucleic acid comprising at least one sequence of consecutive nucleobases capable of hybridizing to at least one nucleic acid strand even if less than all nucleobases do not base pair with a counterpart nucleobase. In certain embodiments, a “substantially complementary” nucleic acid contains at least one sequence in which about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, to about 100%, and any range therein, of the nucleobase sequence is capable of base-pairing with at least one single stranded nucleic acid molecule.

Aspects of the present disclosure are directed to a method of detecting SARS-COV in a sample using a zeolitic imidazolate framework-8 (ZIF-8) based biosensor, or herein referred to as a sensor, to detect the COVID-19. ZIF-8 works as a fluorescence quenching and re-emergence platform to detect the COVID-19 RNA sequences. The sensor of the present disclosure is sensitive and can distinguish a conserved single-strand RNA sequence at 200 pM concentrations. Further, the biosensor can distinguish down to the single mismatch nucleotide in RNA sequences.

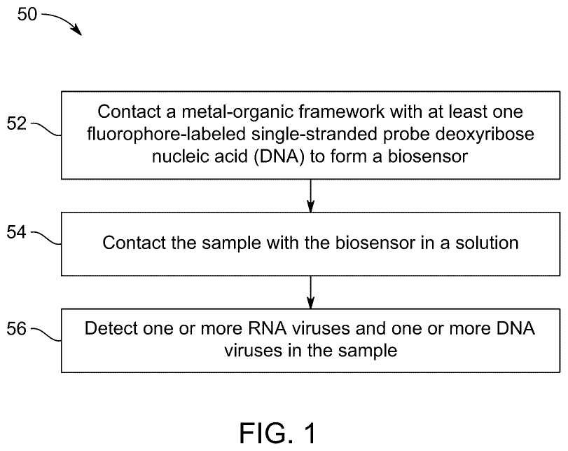

FIG. 1 illustrates a flow chart of method 50 of detecting SARS-COV in a sample. The order in which the method 50 is described is not intended to be construed as a limitation, and any number of the described method steps can be combined in any order to implement the method 50. Additionally, individual steps may be removed or skipped from the method 50 without departing from the spirit and scope of the present disclosure.

At step 52, the method 50 includes contacting a metal-organic framework with at least one fluorophore-labeled single-stranded probe deoxyribose nucleic acid (DNA) to form a biosensor. The metal-organic frameworks (MOFs) have an open structure, multi-porosity, and chemical and thermal stability ideal for sensitive detection and sensing purposes. MOFs are compounds having a lattice structure made from (i) a cluster of metal ions (secondary building units or SBUs) as vertices (cornerstones) which are metal-based inorganic groups, for example metal oxides and/or hydroxides, linked together by (ii) organic ligands. The metal ions may have an oxidation state of 1+, 2+, 3+, 4+, 5+, 6+, and 7+. The metal ions my include, but are not limited to, magnesium, titanium, vanadium, chromium, manganese, iron, cobalt, nickel, copper, zinc, zirconium, silver, cadmium, a combination thereof, and any metal ion known in the art. The organic ligands may be a 2-methylimidazole, a benzene dicarboxylic acid, a dihydroxyterephthalic acid, a trimesic acid, a terephthalate, a benzene tricarboxylic acid, a biphenyldicarboxylate, and any organic ligand know in the art. The organic ligands may be substituted and/or otherwise functionalized with functional groups. The functional groups may include alkanes, alkene, alkynes, hydroxides, carboxylic acids, ethers, ketones, amines, amides, nitrates, nitrides, sulfonates, phosphides, a combination thereof, and any functional groups known in the art. The organic ligands, also called linkers, are usually at least bidentate ligands which coordinate to the metal-based inorganic groups via functional groups such as carboxylates and/or amines. MOFs are considered coordination polymers made up of (i) the metal ion clusters and (ii) linker building blocks. MOFs are a coordinated network of organic ligands with voids or spaces and may form one-, two-, or three-dimensional structures. Additionally, the MOFs can be used as fluorescent quenchers to realize the fluorescence detection of a target molecule due to a π-rich organic ligand or linker and/or a partial coordination of metal ion by π-π stacking and/or electrostatic interaction with fluorophore marked nucleic acid probes. Examples of MOFs include, but are not limited to, ZIF-4, ZIF-8, ZIF-62, ZIF-67, HKUST-1, MOF-5, MOF-76, MOF-177, MOF-200, MOF-210, MIL-53, MIL-88A, MIL-100, MIL-127, UiO-66, UiO-66-NH2, PCN-61, and any MOF known in the art.

In a preferred embodiment, the metal-organic framework is in the form of zeolitic imidazolate framework-8 (ZIF-8). Zeolitic imidazolate frameworks (ZIFs) are comprised of tetrahedrally-coordinated transition metal ions connected by imidazolate linkers. The transition metal ions may include copper, iron, cobalt, zinc, the like, and a combination thereof. ZIFs have high thermal and chemical stability. Especially, ZIF-8 shows high thermal stability found in only a few relatively dense MOF structures. Thus, the ZIF-8 of the present disclosure is at least thermally stable at 450° C., 460° C., 470° C., 480° C., 490° C., 500° C., or at 510° C. In a preferred embodiment, the ZIF-8 has a thermogravimetric analysis stability of up to 510° C. Furthermore, in some embodiments, the ZIF-8 platform is sensitive such that the ZIF-8 with the probe-DNA (p-DNA) may distinguish the target sequence of SARS-COV from within a mixture of several sequences.

In an exemplary embodiment, the ZIF-8 comprises zinc and 2-methylimidazole synthesized at room temperature with at least 75% yield based on a starting weight of zinc salt and 2-methylimidazole. In an embodiment, a zinc salt is dissolved in an organic or in an inorganic solvent. The zinc salt may be a hydrate or an anhydrate salt. The zinc salt may be zinc nitrate, zinc acetate, zinc carbonate, zinc sulfate, zinc sulfide, zinc phosphate, zinc chromate, zinc oxide, zinc chloride, a combination thereof, and any zinc salt known in the art. In a preferred embodiment, the zinc salt is zinc nitrate hexahydrate. In a preferred embodiment, the zinc nitrate hexahydrate is dissolved in water instead of an organic solvent reducing the environmental impact and cost of the synthesis. In one embodiment, the water is distilled or deionized (DI) water. In an embodiment, zinc nitrate hexahydrate is dissolved in DI water to form a zinc solution. In an alternate embodiment, the imidazole is one or more of benzimidazole, 2-ethylimidazole, 2-propylimidazole, or other imidazole derivatives. In an embodiment, triethylamine is added to DI water to form a base solution. In an embodiment, 2-methylimidazole is added to the base solution to form a linker solution. In a preferred embodiment, the zinc solution is added to the linker solution to form a reaction mixture. The reaction mixture is stirred for 1 to 60 minutes, preferably 5 to 20 minutes, and more preferably about 10 minutes. The reaction mixture may be stirred by hand, with a stir plate and a magnetic stir bar, a vortex mixer, an orbital shaker, a platform shaker, and the like. In some embodiments, after the reaction is stirred, the reaction mixture is then centrifuged to remove a supernatant. A remaining solid portion is mixed with DI water to form a suspension. The suspension is mixed for a further 12 to 48 hours, preferably 18 to 36 hours, and more preferably about 24 hours. The suspension is centrifuged with DI water followed by an alcohol. The solids may be first centrifuged 1 to 10 times, preferably 1 to 5 times, and more preferably 1 to 3 times with DI water. The solids may be secondly centrifuged 1 to 10 times, preferably 1 to 5 times, and more preferably 1 to 3 times with the alcohol. In a preferred embodiment, the alcohol is ethanol. The solids are dried at a temperature of 75 to 150° C., preferably 100 to 125° C., and more preferably about 110° C. for 1 to 48 hours, preferably 12 to 36 hours, and more preferably about 24 hours to form a powder. The drying may occur in an oven. The powder is dried under vacuum at a temperature of 100 to 200° C., preferably 125 to 175° C., and more preferably about 150° C. for a time of 30 to 180 minutes, preferably 45 to 150 minutes, and more preferably about 60 to 90 minutes to form the ZIF-8 MOF. In some embodiments, the yield of the ZIF-8 is at least 30%, at least 35%, at least 40%, at least 45%, at least 50%, at least 55%, at least 60%, at least 65%, at least 70% to 74%, and up to about 80% based on the starting weight of the zinc nitrate and 2-methylimidazole.

In certain embodiments, the solution of the method of the present disclosure includes a buffer solution. In some examples, the buffer solution includes a mixture of acids and bases to maintain the pH of the solution. The buffer solution is selected from a group consisting of TAE (TAE or TE), TBE, the like, or a combination thereof. In some specific embodiments, the method 50 includes the solution of a TAE buffer. The TAE buffer includes a tris(hydroxymethyl)aminomethane (Tris) base, an acetic acid, and an ethylenediaminetetraacetic acid (EDTA). In a preferred embodiment of the present disclosure, the TAE buffer is prepared by adding Tris base and EDTA to distilled water. The prepared solution is further diluted with the addition of distilled water, followed by adding an acid to maintain the pH of the prepared buffer solution. In some examples, the acid is selected from HCl, acetic acid, boric acid, the like, or a combination thereof. In a specific embodiment, the acetic acid used is a glacial acetic acid. The pH of the buffer solution may be 8.0 to 9.0, preferably 8.2 to 8.5, and more preferably about 8.3. The prepared buffer solution may be sterilized by autoclaving at a pressure of 10 to 25 psi, preferably 12 to 20 psi, and more preferably about 15 psi for a time of 5 to 90 minutes, preferably 10 to 60 minutes, and more preferably about 20 minutes. The prepared buffer solution may be stored at room temperature before use.

In an embodiment, the ZIF-8 is contacted with one or more fluorophore-labeled single-stranded probe DNA (p-DNA). The fluorophore-labeled single-stranded p-DNA has a fluorescence signal at 513 to 517 nm. The fluorophore-labeled single-stranded p-DNA docks with the metal-organic framework through attractive noncovalent π-orbital overlaps to form the biosensor. A strength of docking between the fluorophore-labeled single-stranded p-DNA and the MOF may depend on size, shape, composition, interaction, and fit of the fluorophore-labeled single-stranded p-DNA and the MOF. In some embodiments, the fluorophore-labeled single-stranded p-DNA docks with the metal-organic framework through pi (π) stacking. Pi stacking, also called π-π stacking, refers to attractive, noncovalent pi interactions (orbital overlap) between the pi bonds of aromatic rings for an electrostatic interaction. Pi stacking may be a sandwich interaction, in which aromatic rings are directly stacked on one another; a perpendicular T-shaped interaction, in which a first aromatic ring intersects a second aromatic ring in a perpendicular plane; and/or a staggered stacking interaction (parallel displaced), in which a first aromatic ring is stacked, not directly, on a second aromatic ring. In the staggered stacking interaction, the aromatic rings may be displaced in one or more parallel planes. The staggered stacking interaction may displace the first aromatic ring a quarter, half, or three-quarters way through the second aromatic ring, and/or the first aromatic ring may be displaced an entire length of the second aromatic ring. The docking may be through a van der Waals force and the like. In an embodiment, the fluorophore-labeled single-stranded p-DNA docks with the metal-organic framework through attractive, noncovalent π-orbital overlaps of at least one imidazole of the 2-methylimidazole of the metal-organic framework and at least one aromatic ring of at least one fluorophore of the fluorophore-labeled single-stranded p-DNA. In some embodiments, a ratio of the fluorophore-labeled single-stranded p-DNA to the ZIF-8 in the biosensor is from 1:1 to 1:100, preferably 1:20 to 1:50, and more preferably about 1:30 by weight. The biosensor is fluorescently inactive. Fluorescently inactive may include a fluorescence signal that is less than 10%, preferably less than 8%, and more preferably less than 5% of the intensity of an initial fluorescence signal of the fluorophore-labeled single-stranded p-DNA at 513 to 517 nm in the absence of the MOF.

At step 54, the method 50 includes contacting the sample with the biosensor in a solution. In some embodiments, the solution may be a suspension of the biosensor in a liquid. In other embodiments, the solution may represent a composition in which the biosensor is dissolved in a liquid and/or homogeneously dispersed in the liquid. In some other embodiments, the solution may be a heterogenous mixture of the biosensor and the liquid. The sample includes a target sequence of SARS-COV. The target sequence of SARS-COV and the fluorophore-labeled single-stranded probe DNA hybridize to form a double-stranded product. In some embodiments, the fluorophore-labeled single-stranded p-DNA is complementary to the target sequence of SARS-CoV. In an embodiment, complementary pairs of nucleotide bases between the fluorophore-labeled single-stranded p-DNA sequence and the target sequence of SARS-COV may form. In an embodiment, standard (Watson-Crick) complementary base pairs of nucleotide bases may be adenine and thymine, adenine and uracil (in RNA), and cytosine and guanine. In an embodiment, complementary may be no mismatches between nucleotide bases. In other embodiments, the fluorophore-labeled single-stranded p-DNA and the target sequence may have 1, 2, 3, 4, 5, 6, 7, or multiple there of mismatched nucleotide base pairs. A mismatched nucleotide base pair may be adenine and cytosine, adenine and guanine, thymine and cytosine, thymine and guanine, uracil and cytosine, uracil and guanine, or a combination thereof. In an embodiment, the probe is at least 75%, at least 80%, at least 85%, at least 90%, at least 95%, or at least 100% complementary to the target sequence of SARS-COV. For example, if the target sequence of SARS-COV and the fluorophore-labeled single-stranded p-DNA have no mismatches between nucleotide bases, the sample is 100 percent complementary. Percent complementary is measured by the number of complementary pairs of nucleotide bases divided by the total number of nucleotide bases in the fluorophore-labeled single-stranded p-DNA.

In some embodiments, the target sequence of SARS-COV is at least 85% complementary to the fluorophore-labeled single-stranded probe DNA and the re-emergent fluorescence signal at 513 to 517 nm of the fluorophore-labeled single-stranded probe DNA is from 15 to 95% of an initial fluorescence signal at 513 to 517 nm of the fluorophore-labeled single-stranded probe DNA. In a preferred embodiment, the probe is 100% complementary to the target sequence of SARS-CoV avoiding any occurrence of mismatches of nucleotide bases. The fluorophore of the present method is fluorescein or a derivative thereof.

Preferably, the fluorophore is selected from a group of fluorescein derivatives including 2′,7′-dimethoxy-5- and 6-carboxy-4,7-dichlorofluorescein, 2′, 7′-dimethoxy-4′,5′-dichloro-5- and 6-carboxy-4,7-dichlorofluorescein, 1′,2′,7′,8′-dibenzo-5- and 6-carboxy-4,7-dichlorofluorescein, 1′,2′,7′,8′-dibenzo-4′,5′-dichloro-5- and 6-carboxy-4,7-dichlorofluorescein, 2′,7′-dimethoxy-4′,5′-dichloro-5- and 6-carboxyfluorescein, 2′, 7′-dichloro-5 and 6-carboxy-4,7-dichlorofluorescein, and 2′,4′,5′,7′-tetrachloro-5 and 6-carboxy-4,7-dichlorofluorescein, 5- and 6-carboxyfluorescein, 5- and 6-carboxy-4,7-dichlorofluorescein. In some specific examples, a fluorophore of the fluorophore-labeled single-stranded probe DNA is a carboxyfluorescein. In a preferred embodiment, the carboxyfluorescein is a 6-carboxyfluorescein moiety. In some embodiments, the fluorophore of the fluorophore-labeled single-stranded probe DNA is tagged at a 5′ end of the fluorophore-labeled single-stranded probe DNA. In some embodiments, “tagged at” may included bonded at, bonded to, and/or otherwise interacting with, and the like at the 5′ end of the fluorophore-labeled single-stranded probe DNA. In some embodiments, the interaction may be a covalent bond and/or a van der Waals force.

In some embodiments, the target sequence of SARS-COV is an RNA target sequence. In an alternate embodiment, the target sequence of SARS-COV is a structural variant of the RNA. Structural variants are about 50 base pairs and may include deletions, insertions of non-reference sequence or mobile elements, duplication, inversion, and translocations. In some specific embodiments, the structural variation is selected from a group, including deletion, duplication, or inversion. In some preferred embodiments, the target sequence of SARS-COV is a conserved sequence. A conserved sequence is an identical or similar sequence in nucleic acids or proteins across species within a genome. A conserved sequence is a sequence that has been maintained by natural selection. In an embodiment, the conserved sequence may be resistant to genetic mutations and may persist throughout generation and variations of SARS-COV. The method of the present disclosure embodies the salient features of detection of the RNA target sequence from known or unknown variants of the SARS-COV virus. In one embodiment, the flexibility of the present method allows the detection of a mixture of the target RNA sequence of the SARS-COV variants present in the sample. Thus, in an exemplary embodiment, the SARS-COV is selected from the group consisting of SARS-COV-2 variants B.1.1.7, which comprises mutation N501Y; B.1.351, which includes substitution mutations K417N, E484K, and N501Y; B.1.351 (substitution mutations E484K); B.1.351 (substitution mutation K417N); B.1.617 (L452R, and E484Q); P.1 (substitution mutations K417T, E484K, N501Y); B.1.1.7 (which comprises a HV69-70 deletion, a Y144 deletion, and substitution mutations N501Y, A570D, D614G, P681H, T716I, S982A, and D1118H); B.1.351 (substitution mutations L18F, D80A, D215G, a LAL242-244 deletion, and substitution mutations R246I, K417N, E484K, N501Y, D614G, and A701V; B.1.351 (substitution mutations D80A, K417N, E484K, N501Y, D614G, and A701V); P.1 (substitution mutations L18F, 20N, P26S, D138Y, R190S, K417T, E484K, N501Y, D614G, H655Y, T1027I, and V1176F); P.1 (substitution mutations L18F, T20N, P26S, D138Y, R190S, K417T, E484K, N501Y, D614G, H655Y, T10271); and the like. In some examples, the SARS-COV is SARS-COV-2.

Time and temperature are parameters for the successful and accurate detection of SARS-CoV. In some of the embodiments, the sample is contacted with the biosensor in the solution at a temperature of at least 0° C., at least 1° C., at least 1.5° C., at least 2° C., at least 2.5° C., at least 3° C., at least 3.5° C., at least 4° C., at least 4.5° C., at least 5° C., at least 5.5° C., at least 6° C., at least 6.5° C., at least 7° C., at least 7.5° C., at least 8° C., at least 8.5° C., at least 9° C., at least 9.5° C. to 10° C. In a preferred embodiment, the sample is contacted with the biosensor in the solution at a temperature of 0° C. to 10° C. In some other embodiments, the sample is contacted with the biosensor in the solution for a time of at least 20, at least 25, at least 30, and at least 35 to at least 40 minutes. In a preferred embodiment, the sample is contacted with the sample with the biosensor in the solution for a time of 20 to 40 minutes. In a specific example, the method includes contacting the sample with the biosensor in the solution for a time of 20 to 40 minutes at a temperature of 0° C. to 10° C.

At step 56, the method 50 includes detecting the double-stranded product by observing a change in fluorescence. In some embodiments, the change in fluorescence is a re-emergent fluorescence signal at 513 to 517 nm of the fluorophore-labeled single-stranded probe DNA. In an embodiment the fluorescence re-emergence signal at 513 to 517 nm of the double-stranded product is 15 to 95%, preferably 20 to 90%, 30 to 80%, 40 to 70%, or 50 to 60%, of the intensity of the initial fluorescence signal at 513 to 517 nm of the fluorophore-labeled single-stranded probe DNA. Furthermore, the re-emergent fluorescence signal at 513 to 517 nm of the fluorophore-labeled single-stranded p-DNA indicates the presence of SARS-COV in the sample, thereby detecting SARS-COV in the sample.

The method of the present disclosure provides flexibility in detecting the target RNA sequence from any part of the SARS-COV genome. In some preferred embodiments, the fluorophore-labeled single-stranded probe DNA comprises SEQ ID NO.: 1 (5′-AGATGTCTTGTGCTGCCGGTA-3′).

The detection of SARS-COV in general, is a health challenge. Rapid and accurate detection has been shown over the years, as seen during a recent worldwide pandemic. In such a scenario, the clinical and epidemiologic impact of the method of detection becomes critical. One parameter that has proved difficult and resulted in loss of lives due to failure to detect the infection present at a lower concentration is an assay with a high limit of detection (LoD). Limit of detection is the lowest signal, or the lowest corresponding quantity (i.e., concentration, absolute amount) to be determined or extracted from the signal, that can be observed with a sufficient degree of confidence or statistical significance. In an embodiment, LoD is the lowest detectable concentration of viral RNA that returns a positive result in ≥95% of repeat measurements. Thus, a high LoD requires more copies of viral RNA per sample to identify a positive case, resulting in a high proportion of false negative results for people who are at the beginning of an infection or are asymptomatic. Therefore, a feature to determine the quality of a method or assay of detecting the SARS-COV in a sample is LoD. Thus, in some embodiments of the present method, the lower limit of detection of the SARS-COV is a concentration of at least 100 pM, at least 105 PM at least 110 pM, at least 115 pM, at least 120 pM, at least 125 pM, at least 130 pM, at least 135 pM, at least 140 pM, at least 145 pM, at least 150 pM, at least 155 pM, at least 160 pM, at least 165 pM, at least 170 pM, at least 175 pM, at least 180 pM, at least 185 pM, at least 190 pM, at least 195 pM, or at least up to 200 pM of the target sequence of SARS-COV in the sample. In a specific embodiment, the lower limit of detection of the SARS-COV is a concentration of 200 pM of the target sequence of SARS-COV in the sample.

In some embodiments, the biosensor of the present disclosure has at least about 90%, at least 91%, at least 92%, at least 93%, at least 94%, at least 95%, at least 96%, at least 97%, at least 98%, at least 99%, or at least about 100% specificity towards SARS-COV virus. Percent specificity is a measure of how well a test can identify true negatives in the sample. Specificity, or a true negative rate, is a measure of the probability of a negative test result, conditioned on the sample truly being negative. For example, if the biosensor of the present disclosure has a 100% specificity towards SARS-COV virus, the biosensor would detect no false positive cases; i.e., samples without a target sequence of SARS-COV would not be falsely identified as containing a target sequence of SARS-COV. For further reference on specificity, see T. Fawcett, “An introduction to ROC analysis”, 2006, 27, 8, 861-874, incorporated herein by reference in its entirety. In some embodiments, the method permits at least about 90%, at least 91%, at least 92%, at least 93%, at least 94%, at least 95%, at least 96%, at least 97%, at least 98%, at least 99%, or at least about 100% sensitivity towards SARS-COV virus.

In some aspects of the method of the present disclosure, the method includes detecting one or more RNA viruses and one or more DNA viruses in the sample. In some embodiments, the method may detect the presence of the RNA and/or DNA viruses in the sample among a mixture of nucleic acid sequences. In some embodiments, the method detects one or more RNA viruses and one or more DNA viruses in the sample from the group comprising single- and double-stranded RNA and/or, single- and double-stranded DNA viruses. Some examples of the viruses include, but not limited to, arenaviruses, bunyaviruses, flaviviruses, coronaviruses, togaviruses, filoviruses, paramyxoviruses, rhabdoviruses, reoviruses, adenoviruses, herpesviruses, polyomaviruses, poxviruses, or any combination of viruses of interest.

In one embodiment, the detection does not require a detection device. In another embodiment, the biosensor is embedded into a detection device such as, example, a glucometer, or a mobile phone. The time to result provided by the method may vary. In one embodiment, the detection of SARS-COV occurs within about 60 minutes or less, about 50 minutes or less, about 40 minutes or less, about 30 minutes or less, about 10 minutes or less, about 5 minutes or less, about 2 minutes or less, or about 1 minute or less. In a particular embodiment, the time to the detection is less than 1 minute or more particularly, about 30 seconds, about 20 seconds, about 10 seconds, or about 1 second.

In some embodiments, the method of the present disclosure can also be used as a kit or a part of a reagent box for real-time or clinical detection of viruses of interest. The kit or the reagent box may include the dosages of the biosensor, the fluorophore-labeled single-stranded probe, and the solution or the reagents of the method for performing the detection of the viruses of interest.

### Examples

The disclosure will now be illustrated with working examples, which are intended to illustrate the working of disclosure and not intended to restrictively imply any limitations on the scope of the present disclosure, as many variations thereof are possible without departing from the spirit and scope of the present disclosure. The working examples depict an example of the method of the present disclosure.

### Materials

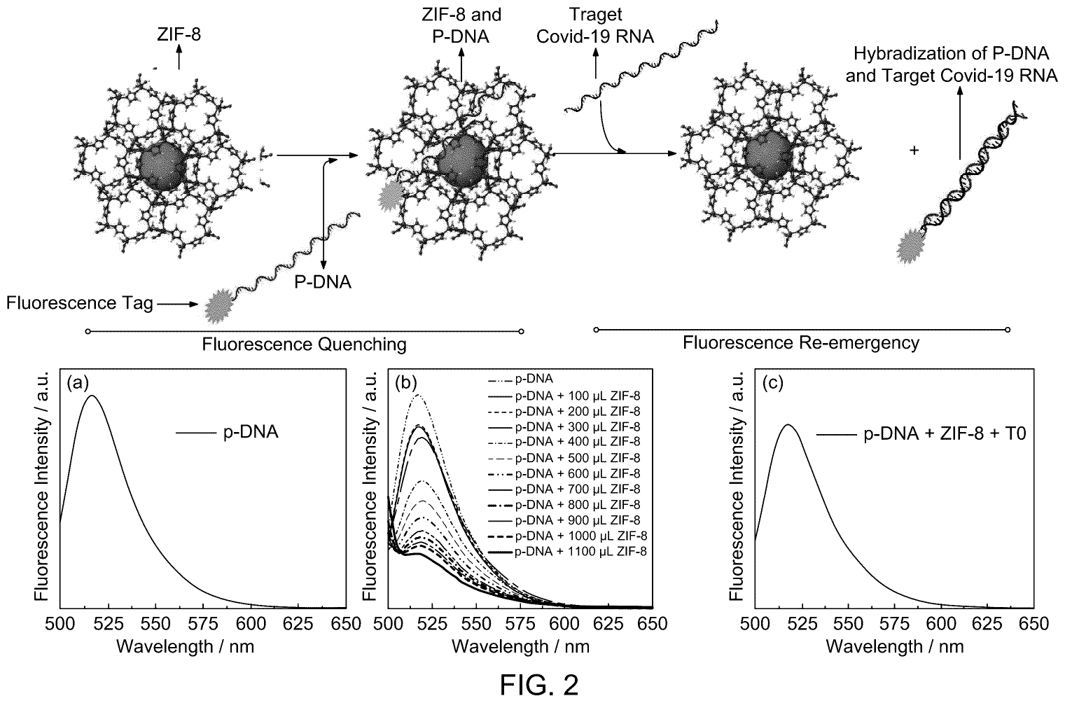

The materials used for the examples and embodiments of the present method include Zn(NO₃)₂·6H₂O (98%, Sigma-Aldrich), Co(NO₃)₂·6H₂O (98%, Sigma-Aldrich), and 2-methylimidazole (97%, Sigma-Aldrich) obtained from commercial sources and used without further purification. Referring to FIG. 2, the coronavirus (COVID-19) conserved sequences, 21 base pairs (bp), were determined through Clustered Omega bioinformatic software and further confirmation was done through NCBI BLAST against genomes of possible strains of COVID-19.

The sequence was unique in all strains of COVID-19. All COVID-19 conserved sequences, Probe-DNA (p-DNA), and RNA with one and two mismatches were commercially obtained (Microgen, Inc, South Korea). The sequence of probe-DNA was (p-DNA) 5′-AGATGTCTTGTGCTGCCGGTA-3′ with 6-carboxyfluorescein (FAM)-labeled at 5′-terminal. Sequence of COVID-19 RNA (TO) was, TO: 5′-UACCGGCAGCACAAGACAUCU-3′, which is 100 percent complementary to the p-DNA; sequences with one mismatch, T1: 5′-UACCGGCAGCACCAGACAUCU-3′ and with two mismatches, T2: 5′-UACCGGCAGCACCAGACGUCU-3′. All the RNA sequences were suspended in Tris buffer solution and placed at −80° C. The Probe-DNA was placed at 4° C. All instruments employed for the development of COVID-19 were autoclaved and disinfected. Fluorescence spectra were measured using a Jasco Spectrophotometer FP-8500 equipped with a xenon discharge lamp and 1 cm quartz cells with a slit width of 2 nm for the source and the detector. Powder X-ray diffraction (PXRD) was carried out by the Rigaku 2500 VBZ+/PC instrument. Thermogravimetric analysis (TGA) was carried out on a PerkinElmer Pyris Diamond TG Thermogravimetric Analyzer at a heating rate of 10° C. min-1 [Analytical Biochemistry, 2018. 546: p. 5-9, incorporated by reference herein in its entirety].

### Synthesis of ZIF-8 MOF

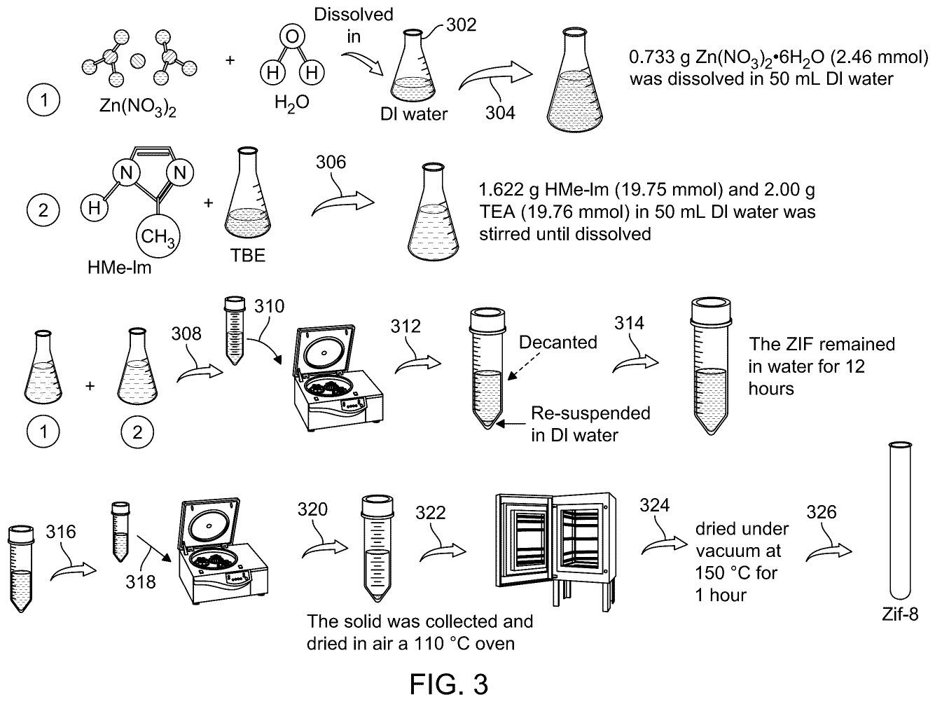

Referring to FIG. 3, an exemplary embodiment of preparing the ZIF-8 MOF is illustrated. At step 302, as can be observed from FIG. 3, the method includes dissolving 0.733 g of Zn(NO₃)₂·6H₂O (2.46 mmol) in 50 mL of deionized water (302) to form a zinc solution (304). Further, a solution of 2-methylimidazole (Hme-Im) and triethylamine (TEA) was formed by taking 1.622 g Hme-Im (19.75 mmol) and 2.00 g of TEA (19.76 mmol) in 50 mL deionized water and stirring till a clear, Hme-Im/TEA solution is formed (306). At step 308, the zinc solution (as prepared in step 304) was added to the stirred Hme-Im/TEA solution (as prepared in step 306), followed by stirring for 10 minutes. The resultant solution is centrifugated to remove the supernatant leaving a solid portion in the mixture (310). The supernatant was decanted, and the solid portion was resuspended in deionized water (312) and placed overnight for about 12 hours (314) to yield ZIF (316). This is followed by centrifugation first with deionized water (318) and then with ethanol (320). Furthermore, the solid portion from the centrifugation is dehydrated in an oven at about 110° C. (322), and the resultant powder is collected. The resultant powder is further dried under vacuum at 150° C. for about 1 hour (324) before collecting the synthesized ZIF-8 MOF (326).

### Preparation of TAE Buffer

For the preparation of TAE buffer, the following protocol was adopted. 1.576 g of the Tris base and 0.292 g of the EDTA were weighed. 80 mL of distilled water was added to an autoclaved flask. The weighed Tris base and the EDTA were added to the autoclaved flask with the distilled water. I resultant solution was brought up to 100 mL with distilled water. The pH of the resultant solution was obtained and maintained via HCl addition using a pH meter and adding HCl as needed. The prepared TAE buffer solution is autoclaved and stored at room temperature.

### Measurements

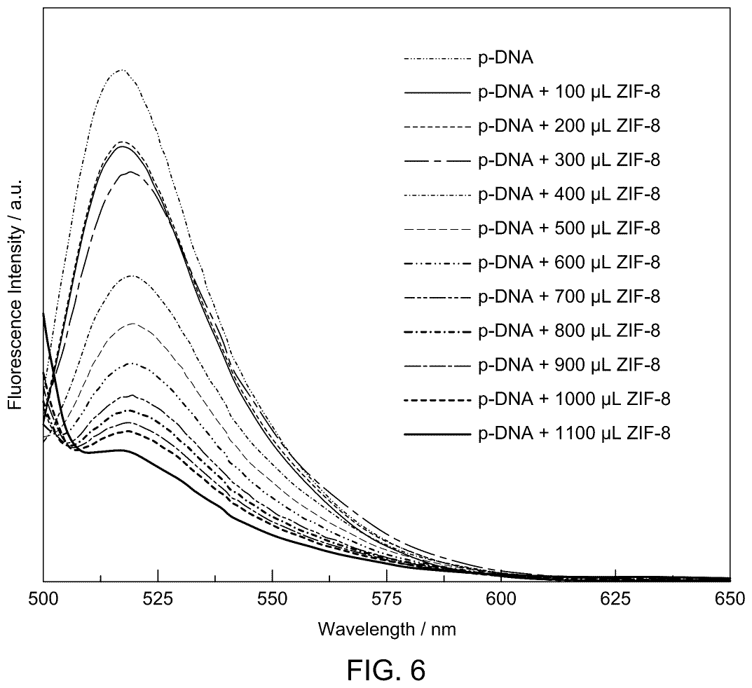

1100 μL Zeolitic imidazolate frameworks 8 (ZIF-8) quenched the fluorescence of FAM-tagged p-DNA (See FIG. 6). 900 μL of (100 nM) of FAM-tagged probe DNA (p-DNA) and 1100 μL of ZIF-8 (1 mg/mL) were added sequentially into a first 2 mL tube for 60 minutes at room temperature. The no fluorescence signal was detected from 513 to 517 nm. Then, 1 mL from the first 2 mL tube and 200 μL of targeted TO COVID-19 RNA (200 pM) were sequentially added into a new 2 mL tube. After incubation on ice for 30 minutes, the fluorescence signal at 513 to 517 nm was detected.

### Characterization of Synthesized Zeolitic Imidazolate Framework-8 (ZIF-8)

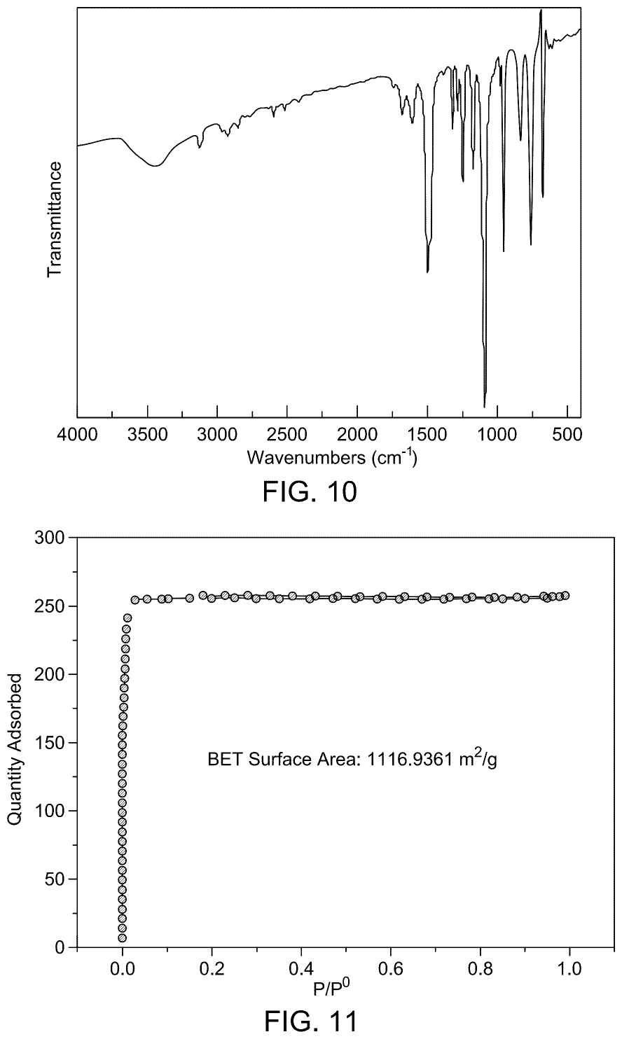

ZIF-8 was synthesized with a 79% yield from the reaction of zinc nitrate hexahydrate and 2-methyleimdazole in the de-ionized H2O at room temperature. The prepared ZIF-8 was further characterized through X-ray diffraction (XRD), TGA, Fourier transform infrared (FTIR) spectroscopy, N2 adsorption, and field emission scanning electron spectroscopy (FESEM). The IR data (FIG. 10) supported that the bands at 3135 and 2929 cm-1 are due to the aromatic and the aliphatic C—H stretch of the imidazole. The peak at 1584 cm-1 supports a C═N stretching mode, whereas the intense and convoluted bands at 1350-1500 cm-1 are associated with the entire imidazole ring stretching. The bands in the spectral region of 900-1350 cm-1 are suggestive of in-plane bending of the imidazole ring, while those below 800 cm-1 are assigned as out-of-plane bending.

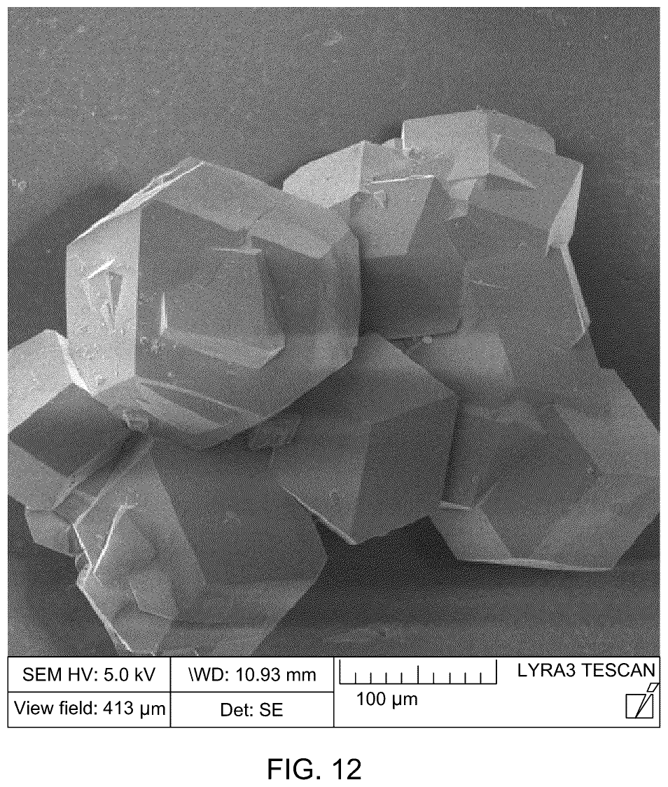

The N₂ adsorption isotherm data (FIG. 11) support a Brunauer-Emmett-Teller (BET) surface area of 1116.9361 m²/g. The FESEM data (FIG. 12) show a conglomeration of three-dimensional cubic structures of the ZIF-8. The cubic structures may be hexagonal and the like. The 3D structures have straight edges. The structure may have edges in length from 25-300 μm, preferably 50-200 μm, and more preferably about 75-150 μm.

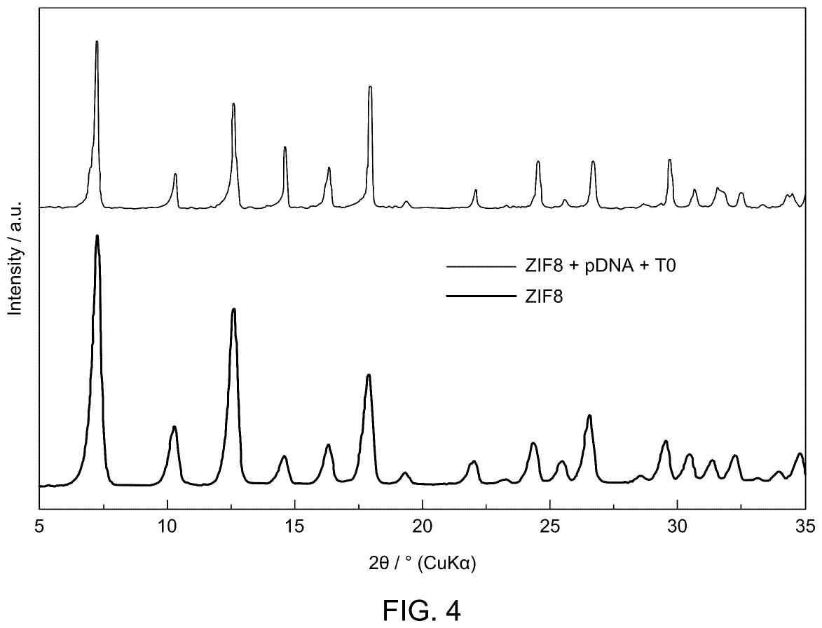

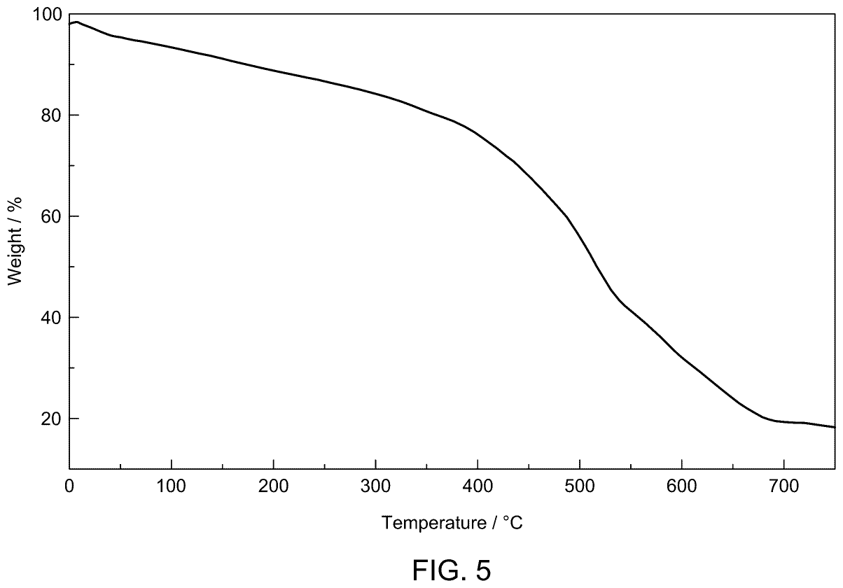

The ZIF-8 was dissolved in TAE buffer with p-DNA and complementary T0 RNA sequences for 2 days. Later, the XRD of the ZIF-8+p-DNA_T0 was compared with simple ZIF-8. There was no structural difference examined in the peaks of only ZIF-8 and ZIF-8+p-DNA+T0 (FIG. 4). The TGA analysis of prepared ZIF-8 was done. The TGA analysis showed that the ZIF-8 is stable until 510° C. (FIG. 5).

TABLE 1
X-ray diffraction (XRD) analyses results

| Peak No. | 2-theta (deg) | Height(cps) |
|---|---|---|
| 1 | 8.075(5) | 2269(61) |
| 2 | 11.059(6) | 1522(50) |
| 3 | 13.383(4) | 4403(86) |
| 4 | 15.341(8) | 1111(43) |
| 5 | 17.084(4) | 2947(70) |
| 6 | 18.658(4) | 3004(71) |
| 7 | 20.07(3) | 115(14) |
| 8 | 22.698(13) | 377(25) |

### Biosensing Properties of ZIF-8 Toward the Coronavirus (COVID-19) RNA Sequences

To understand the biosensing properties of the ZIF-8 MOF synthesized with 2-methylimidazole as a linker, the structure of the ZIF-8 is looked at. The imidazole contains a π-electron system with an ability to interact with the DNA dye, fluorophore 6-carboxyfluorescein (FAM), tagged at 5′ end of single stranded p-DNA to form π-π stacking that quenches the fluorescence. Referring to FIG. 6, the ability of ZIF-8 MOF to bind with the fluorophore tagged p-DNA and quench the fluorescence signal was tested by sequentially adding 100 μL aliquots of ZIF-8 to the solution of FAM containing p-DNA: FAM-5′-AGATGTCTTGTGCTGCCGGTA-3′(SEQ ID NO.: 1). Gradually, the fluorescence of p-DNA decreased until 1100 ul of 1 mg/mL ZIF-8 was added into 1 mL of p-DNA solution in the TAE buffer. Through experiments, the quenching was recorded to take 60 minutes. Thus, indicating the quenching of fluorescence was via T-T stacking.

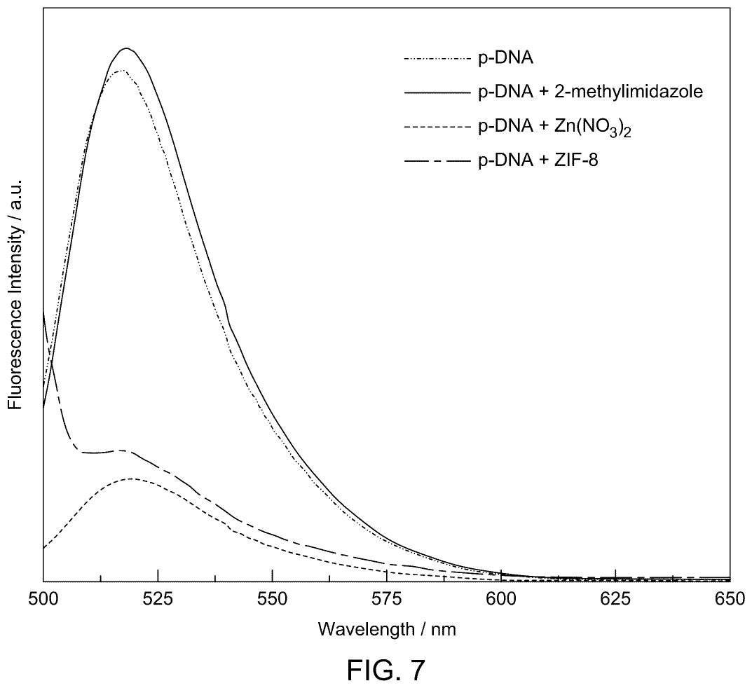

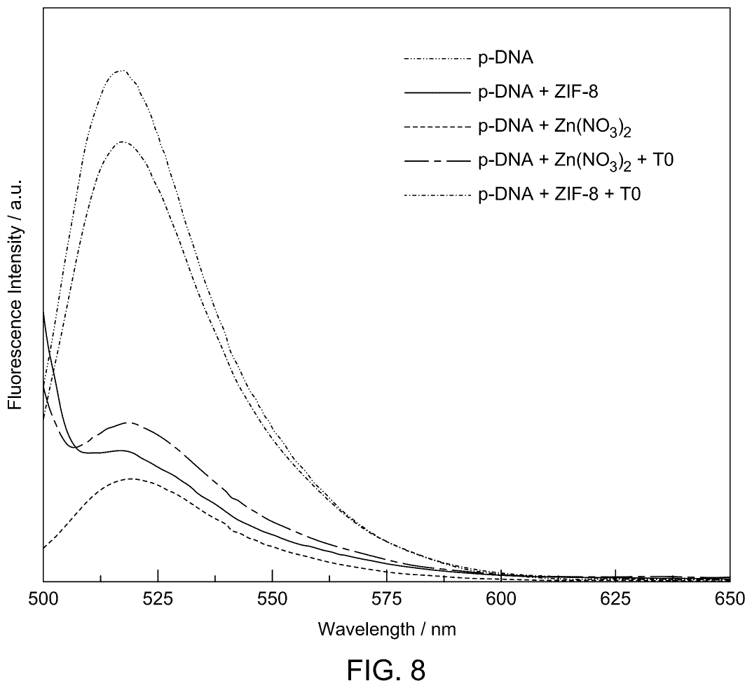

Referring to FIG. 7, to further investigate the quenching process, the p-DNA in the TAE buffer solution was tested individually against the zinc salt, zinc nitrate, and the linker, 2-methyleimidalzole. The linker, 2-methyleimidazole, had a negligible effect on fluorescence quenching. In contrast to the linker, the zinc nitrate quenched the fluorescence signal more than the ZIF-8. These results indicate that the Zn(NO₃)₂ can dimmish the fluorescence signal of the p-DNA. Intercalation of zinc ions into the nucleotides of p-DNA or a photoinduced electron transfer (PET) from the fluorescence tag to zinc ions may have supported the quenching of the fluorescence signal of the p-DNA. This further supports ZIF-8 can quench the fluorescence of the p-DNA through the π-π stacking and PET. Coronavirus RNA with a TO sequence, TO: 5′-UACCGGCAGCACAAGACAUCU-3′, 100% complementary to the p-DNA was added to the p-DNA/ZIF-8 complex and a 90 percent peak re-emergence is illustrated in FIG. 8. The re-emergence occurs from the duplex formation of the TO RNA and the p-DNA. An RNA/DNA duplex forms that causes the p-DNA to be dislocated from the ZIF-8 [Chemical Physics Impact, 2020. 1: p. 100001, incorporated by reference herein in its entirety]. The RNA/DNA duplex forms hydrogen bonds between the single-stranded p-DNA and the RNA sequence, which is a stronger interaction than the π-stacking between the fluorophore of the single-stranded p-DNA and the ZIF-8. Little re-emergence due to strong electrostatic interaction between the p-DNA and FAM was observed. This showing that the highly porous structure of the ZIF-8/p-DNA complex is efficient and can quench the fluorescence signal and then re-generate the fluorescence signal by adding the TO COVID-19 RNA sequence to the ZIF-8/p-DNA complex. The ZIF-8/p-DNA complex can be used a biosensor to detect the conserved, TO RNA sequences of the Coronavirus.

### Single Mismatch Sensitivity

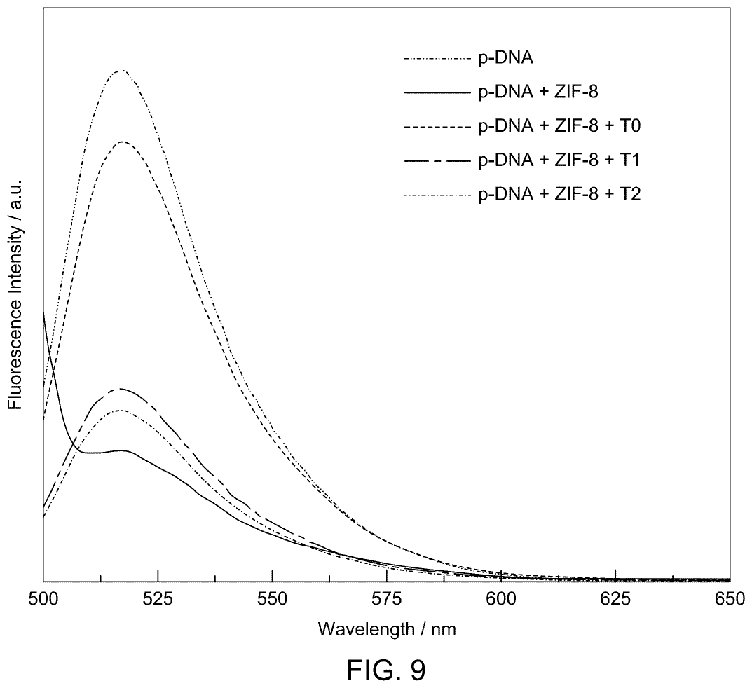

Referring to FIG. 9, further investigation was done of ZIF-8/p-DNA complex biosensor toward COVID-19 RNA sequences with one mismatch, T1: 5′-UACCGGCAGCACCAGACAUCU-3′ and with two mismatches, T2: 5′-UACCGGCAGCACCAGACGUCU-3′ to hybridize with the p-DNA. The fluorescence emergence of COVID-19 RNA TI and T2 sequences was 30 and 20 percent, respectively, compared to TO with 90 percent. Thus, supporting that the ZIF-8/p-DNA complex may be used as a biosensor to detect the COVID-19 through DNA/RNA hybridization.

The method of the present invention disclosure shows several advantages; thus, one of the advantages of the method is that the method is cost-effective and easy to produce. Another advantage of the method of the present invention is that the method is rapid and accurate in detecting the SARS-COV. Yet another advantage of the present method is that the method is flexible and can be provided as part of a reagent box or a kit to perform the detection of the SARS-CoV or any other viruses of interest. Therefore, a notable advantage of the biosensor and the method disclosed here can be employed to detect future pandemic viruses.

Numerous modifications and variations of the present disclosure are possible in light of the above teachings. It is, therefore, to be understood that within the scope of the appended claims, the disclosure may be practiced otherwise than as specifically described herein.

1: A method of detecting SARS-COV in a sample, comprising:
contacting a metal-organic framework with at least one fluorophore-labeled single-stranded probe deoxyribose nucleic acid (DNA) to form a biosensor,
wherein the metal-organic framework is zeolitic imidazolate framework-8 (ZIF-8),
wherein the fluorophore-labeled single-stranded probe DNA has a fluorescence signal at 513 to 517 nm in the absence of the ZIF-8,
wherein the fluorophore-labeled single-stranded probe DNA docks with the metal-organic framework through attractive noncovalent π-orbital overlaps,
wherein the biosensor is fluorescently inactive,
contacting the sample with the biosensor in a solution,
wherein the sample comprises a target sequence of SARS-COV,
wherein the target sequence of SARS-COV and the fluorophore-labeled single-stranded probe DNA hybridize to form a double-stranded product,
detecting the double-stranded product by observing a change in fluorescence,
wherein the change in fluorescence is a re-emergent fluorescence signal at 513 to 517 nm of the fluorophore-labeled single-stranded probe DNA,
wherein the re-emergent fluorescence signal at 513 to 517 nm of the fluorophore-labeled single-stranded probe DNA indicates the presence of SARS-COV in the sample, thereby detecting SARS-COV in the sample.

2: The method of claim 1, wherein the fluorophore-labeled single-stranded probe DNA is 100 percent complementary to the target sequence of SARS-COV.

3: The method of claim 1, wherein a fluorophore of the fluorophore-labeled single-stranded probe DNA is a carboxyfluorescein.

4: The method of claim 3, wherein the carboxyfluorescein is a 6-carboxyfluorescein moiety.

5: The method of claim 3, wherein the fluorophore of the fluorophore-labeled single-stranded probe DNA is tagged at a 5′ end of the fluorophore-labeled single-stranded probe DNA.

6: The method of claim 1, wherein the target sequence of SARS-COV is an RNA target sequence.

7: The method of claim 1, wherein the target sequence of SARS-COV is a conserved sequence.

8: The method of claim 1, wherein the SARS-COV is SARS-COV-2.

9: The method of claim 1, wherein the ZIF-8 comprises zinc and 2-methylimidazole and was synthesized at room temperature with an at least 75% yield based on a starting weight of a zinc salt and 2-methylimidazole.

10: The method of claim 1, wherein the ZIF-8 has a thermogravimetric analysis stability of up to 510° C.

11: The method of claim 1, wherein a lower limit of detection of the SARS-COV is a concentration of 200 pM of the target sequence of SARS-COV in the sample.

12: The method of claim 1, wherein the solution comprises a TAE buffer comprising:
a tris(hydroxymethyl)aminomethane base;
an acetic acid, and
an ethylenediaminetetraacetic acid.

13: The method of claim 1, wherein the fluorophore-labeled single-stranded probe DNA docks with the metal-organic framework through attractive noncovalent I-orbital overlaps of at least one imidazole the 2-methylimidazole of the metal-organic framework and at least one fluorophore of the fluorophore-labeled single-stranded probe DNA.

14: The method of claim 1, wherein a ratio of the fluorophore-labeled single-stranded probe DNA to the ZIF-8 in the biosensor is from 1:10 to 1:50 by weight.

15: The method of claim 1, wherein the re-emergent fluorescence signal at 513 to 517 nm of the fluorophore-labeled single-stranded probe DNA is at least 90% of an initial fluorescence signal at 513 to 517 nm of the fluorophore-labeled single-stranded probe DNA in the absence of the ZIF-8.

16: The method of claim 1, wherein the contacting the sample with the biosensor in the solution is at a temperature of 0 to 10° C.

17: The method of claim 1, wherein the contacting the sample with the biosensor in the solution occurs for a time of 20 to 40 minutes.

18: The method of claim 1, wherein the target sequence of SARS-COV is at least 85% complementary to the fluorophore-labeled single-stranded probe DNA and the re-emergent fluorescence signal at 513 to 517 nm of the fluorophore-labeled single-stranded probe DNA is from 15 to 95% of an initial fluorescence signal at 513 to 517 nm of the fluorophore-labeled single-stranded probe DNA in the absence of the ZIF-8.

19: The method of claim 1, wherein fluorophore-labeled single-stranded probe DNA comprises SEQ ID NO.: 1.

20: The method of claim 1, further comprising:
detecting one or more RNA viruses and one or more DNA viruses in the sample.
